(19) United States
(12) Patent Application Publication
(10) Pub. No.: US 2024/0033140 A1
(43) Pub. Date: Feb. 1, 2024

(54) ABSORBENT ARTICLES WITH DISPOSAL FASTENERS HAVING INTEGRAL HOOK FASTENERS

(71) Applicant: The Procter & Gamble Company, Cincinnati, OH (US)

(72) Inventors: Sarah Marie DeCRISTOFARO, Cincinnati, OH (US); Christian Michael BEST, Cincinnati, OH (US)

(21) Appl. No.: 18/358,496

(22) Filed: Jul. 25, 2023

Related U.S. Application Data
(60) Provisional application No. 63/392,873, filed on Jul. 28, 2022.

(51) Int. Cl. A61F 13/551; A61F 13/62; A61F 13/49

(52) U.S. Cl. CPC A61F 13/5512; A61F 13/62; A61F 13/49

(57) ABSTRACT

An absorbent article having at least one disposal fastener. The disposal fastener includes a plurality of hook fasteners that are integral with a material of the absorbent article. The disposal fastener is configured to engage and hold a portion of the garment-facing surface of the absorbent article and secure the article in a compact configuration for disposal after the article has been used.

### CROSS REFERENCE TO RELATED APPLICATION

This application claims the benefit of U.S. Provisional Application No. 63/392,873, filed Jul. 28, 2022, the substance of which is incorporated herein by reference.

### FIELD OF THE INVENTION

The present disclosure relates to absorbent articles with disposal fasteners, in particular, disposal fasteners having a plurality of integrally formed hook fasteners.

### BACKGROUND OF THE INVENTION

Absorbent articles that are wearable by a user, such as diapers, training pants, adult incontinence briefs, and feminine hygiene products, are designed to offer the benefit of receiving and containing urine and/or other bodily exudates (e.g., feces, menses, mixture of feces and urine, mixture of menses and urine, etc.). After use, absorbent articles are usually folded or rolled into a more compact configuration such that the soiled portion is wrapped inside for disposal. Disposal fasteners may be used on a variety of disposable absorbent articles to maintain the compact configuration and to keep the bodily exudates contained within the article prior to disposal. However, current disposal fasteners use hook fastener materials that are made from multiple components, often from multiple suppliers, that must be assembled during manufacture. The hook fastener materials are also quite expensive. This combination of components requires a number of steps in the process of manufacturing which increases the cost and complexity of manufacturing.

There is a need for an absorbent article with a disposal fastener that requires less materials and that can be made without undesirable cost and complexity while still providing sufficient fastening ability to hold the absorbent article in a compact disposal configuration.

### SUMMARY OF THE INVENTION

The present disclosure solves the problems of the undesirable, complex, and costly hook fasteners for disposal fasteners of absorbent articles by providing hook fasteners that are integral with a material of at least a portion of a disposal fastener. The disposal fasteners of the present disclosure having integral hook fasteners are much simpler to manufacture and provide an economic benefit, while still providing excellent performance in a disposal fastener.

An absorbent article comprises a garment-facing surface; a wearer-facing surface; a front waist region; a back waist region; a crotch region positioned intermediate the front waist region and the back waist region; a first side edge; a second side edge opposite the first side edge; a first end edge; a second end edge opposite the first end edge; a liquid permeable topsheet forming a portion of the wearer-facing surface; a liquid impermeable backsheet; an absorbent core comprising an absorbent material positioned at least partially intermediate the topsheet and the backsheet; an acquisition material positioned intermediate the absorbent core and the topsheet; and an outer cover nonwoven material in a face to face relationship with the backsheet and forming a portion of the garment-facing surface. The absorbent article also comprises a disposal fastener, wherein the disposal fastener is a disposal tape comprising a first end region, a second end region, and a central region. The disposal tape comprises a plurality of hook fasteners in the second end region. The plurality of hook fasteners are integral with a material of the second end region of the disposal tape.

An absorbent article comprises a garment-facing surface; a wearer-facing surface; a front waist region; a back waist region; a crotch region positioned intermediate the front waist region and the back waist region; a first side edge; a second side edge opposite the first side edge; a first end edge; a second end edge opposite the first end edge; a liquid permeable topsheet forming a portion of the wearer-facing surface; a liquid impermeable backsheet; an absorbent core comprising an absorbent material positioned at least partially intermediate the topsheet and the backsheet; an acquisition material positioned intermediate the absorbent core and the topsheet; and an outer cover nonwoven material in a face to face relationship with the backsheet and forming a portion of the garment-facing surface. The absorbent article also comprises a first ear extending from the back waist region and comprising a first joining portion and a second ear extending from the front waist region and comprising a second joining portion. The first joining portion of the first ear is configured to be joined to the second joining portion of the second ear to form a refastenable side seam. The first ear or the second ear comprises a disposal fastener, wherein the disposal fastener is a disposal tab extending beyond the first or second joining portion. The disposal tab comprises a fastener region having a plurality of hook fasteners that are integrally formed with a material of the disposal tab.

An absorbent article comprises a garment-facing surface; a wearer-facing surface; a front waist region; a back waist region; a crotch region positioned intermediate the front waist region and the back waist region; a first side edge; a second side edge opposite the first side edge; a first end edge; a second end edge opposite the first end edge; a liquid permeable topsheet forming a portion of the wearer-facing surface; a liquid impermeable backsheet; an absorbent core comprising an absorbent material positioned at least partially intermediate the topsheet and the backsheet; an acquisition material positioned intermediate the absorbent core and the topsheet; and an outer cover nonwoven material in a face to face relationship with the backsheet and forming a portion of the garment-facing surface. The absorbent article also comprises a disposal fastener, wherein the disposal fastener is a disposal patch comprising a plurality of integral hook fasteners formed from a portion of the outer cover nonwoven material. The disposal patch is positioned on the front waist region or the back waist region and is configured to engage and hold a portion of the garment-facing surface of the absorbent article.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4, with a disposal fastener positioned in the back waist region;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is a plan view of an example absorbent article of the present disclosure that is a sanitary napkin;

FIG. 13 is an example cross-sectional view taken within a front waist region of an absorbent article;

FIG. 14 is an example cross-sectional view taken within a crotch region of an absorbent article;

FIG. 15 is an example cross-sectional view taken within a back waist region of an absorbent article;

FIG. 16 is a cross-sectional side view of a system for forming hook fasteners that are integral with a substrate, such as a nonwoven or a film;

FIG. 17 is a cross-sectional view taken within a plurality of hook fasteners, according to one or more embodiments shown and described herein;

FIG. 18 is a side perspective view of an absorbent article with a disposal tab in an unfastened condition, according to one or more embodiments shown and described herein;

FIG. 19 is a plan view of an absorbent article with a disposal tape, according to one or more embodiments shown and described herein;

FIG. 20 is a plan view of an absorbent article with a disposal tape in a disposal configuration, according to one or more embodiments shown and described herein;

FIG. 21 is an enlarged fragmented perspective view of the absorbent article of FIG. 19 illustrating the disposal tape in a partially extended configuration; and

FIG. 22 is a perspective view of the disposal tape of FIG. 21, fastening surface facing the viewer, in an extended configuration.

### DETAILED DESCRIPTION OF THE INVENTION

### Definitions

The following term explanations may be useful in understanding the present disclosure.

As used herein, “absorbent article” refers to devices such as consumer products that absorb and contain body exudates and, more specifically, refers to devices which are placed against or in proximity to the body of the wearer to absorb and contain the various exudates discharged from the body such as soils and wastes. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, sanitary napkins, and the like. Non-limiting examples of absorbent pants include training pants, pull-on or refastenable pant-type diapers, incontinence briefs and undergarments, and the like.

As used herein, “disposable” refers to absorbent articles which generally are not intended to be laundered or otherwise restored or reused as an absorbent article (e.g., they are intended to be discarded after a single use and may also be configured to be recycled, composted, or otherwise disposed of in an environmentally compatible manner. Some embodiments of the absorbent articles described herein are disposable, others maybe reusable or partially reusable.

In regard to structural terms, as used herein, “disposed” means that an element(s) is located in a particular place or position as a macro-unitary structure with other elements or as a separate element joined to another element.

As used herein, the term “joined” encompasses configurations whereby an element is directly secured to another element by affixing the element directly to the other element, and configurations whereby an element is indirectly secured to another element by affixing the element to intermediate member(s), which in turn are affixed to the other element.

As used herein, “integral” means configurations whereby an element is created from or created by an article component, or portions thereof, as opposed to being joined to the component. “Integrally formed” means an element is created from an underlying material or portion thereof, by for example molding, shaping and/or reconstituting the material.

In regard to material terms, as used herein, the term “nonwoven” refers to a material made from continuous (long) filaments (fibers) and/or discontinuous (short) filaments (fibers) by processes such as spunbonding, meltblowing, carding, and the like. In some configurations, a nonwoven may comprise a polyolefin based nonwoven, including but not limited to nonwovens having polypropylene fibers and/or polyethylene fibers and/or bicomponent fibers comprising a polyolefin. Nonlimiting examples of suitable fibers include spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers as known in the art, and workable combinations thereof. Nonwovens do not have a woven or knitted filament pattern.

Various non-limiting forms of the present disclosure will now be described to provide an overall understanding of the principles of the structure, function, manufacture, and use of absorbent articles with disposal fasteners having integral hook fasteners disclosed herein. One or more examples of these non-limiting forms are illustrated in the accompanying drawings. Those of ordinary skill in the art will understand that the absorbent articles with disposal fasteners having integral hook fasteners described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

The present disclosure relates to absorbent articles having one or more disposal fasteners that may engage with a portion of the outer cover of the absorbent article to hold the used article in a compact configuration for disposal and to prevent bodily exudates from seeping out. The disposal fastener may comprise a plurality of hook fasteners that are integral with a substrate of the absorbent article, such as a polymer nonwoven. The hook fasteners may be made by (i) disposing a substrate between a sonotrode and a proximal surface of a tool defining a plurality of negative molds of hook fasteners, (ii) delivering energy to the substrate such that a temperature of the first side of the substrate increases above the polymer's glass transition temperature, and (iii) compressing the substrate between the sonotrode and the tool such that the polymer of the substrate flows into the cavities until the polymer substantially fills the cavities to form a plurality of hook fasteners on the first side of the substrate that are integral with the substrate.

The disposal fasteners described herein may be present on a variety of absorbent articles.

### General Description of an Absorbent Article

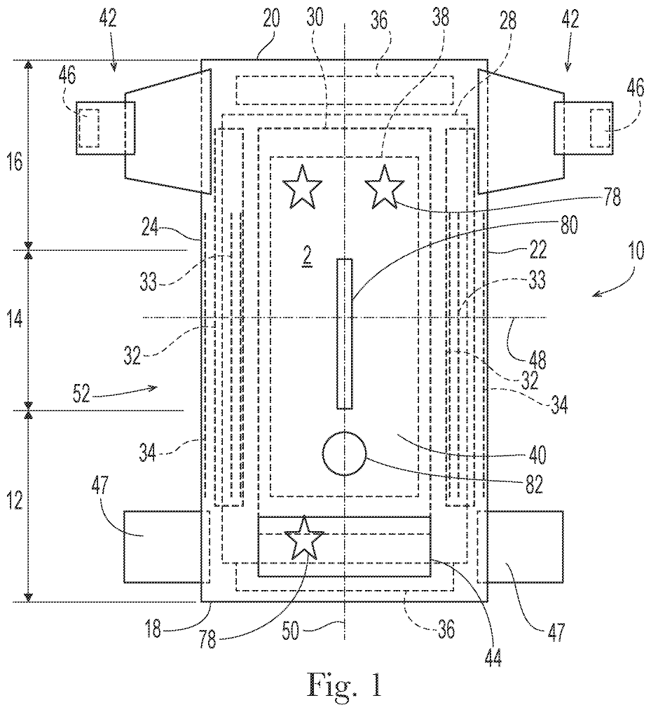

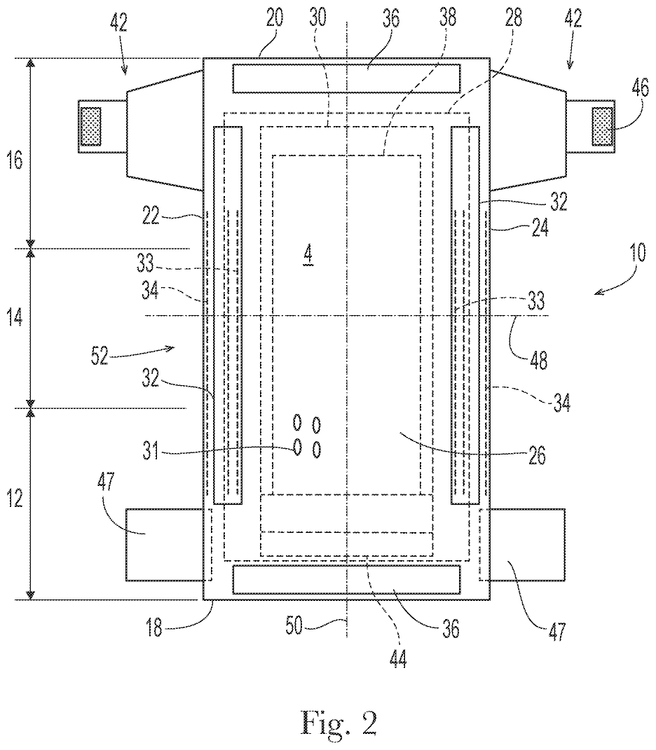

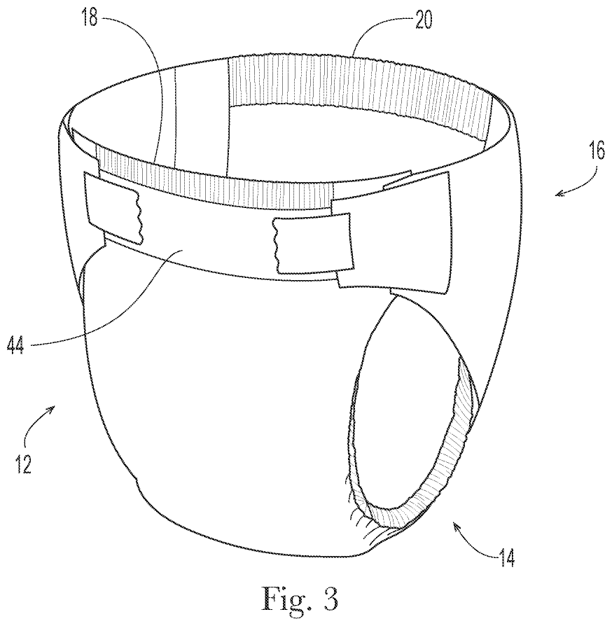

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front waist region 12, the crotch region 14, and the back waist region 16 may each be ⅓ of the length of the absorbent article 10. The absorbent article 10 may comprise a front end edge 18 (also referred to herein as a first end edge), a back end edge 20 (also referred to herein as a second end edge) opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article may comprise back ears 42 (also referred to herein as a first ear or a third ear) in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 (also referred to herein as a second ear or a fourth ear) in the front waist region 12. The absorbent article 10 may have a central lateral (or transverse) axis 48 and a central longitudinal axis 50. The central lateral axis 48 extends perpendicular to the central longitudinal axis 50.

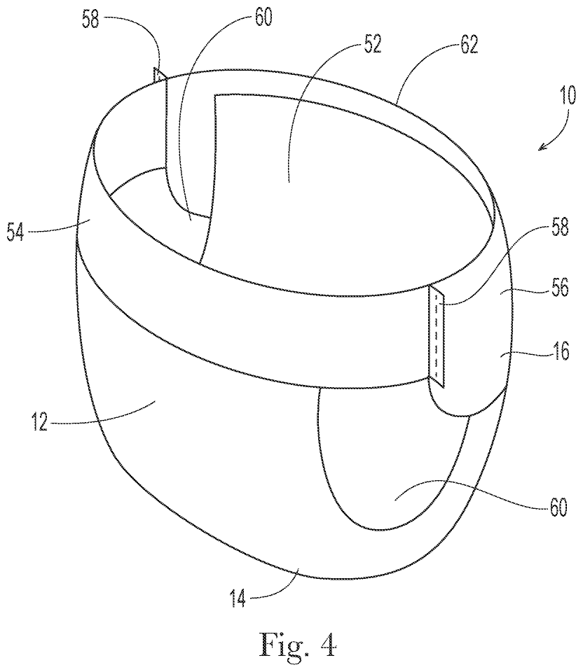

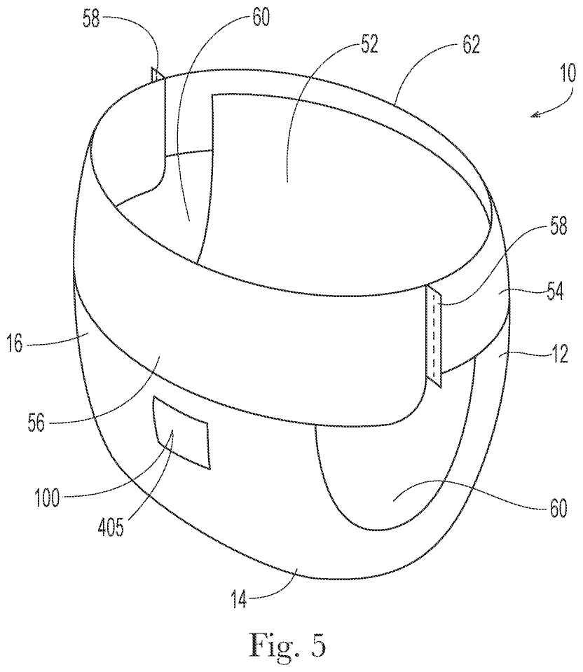

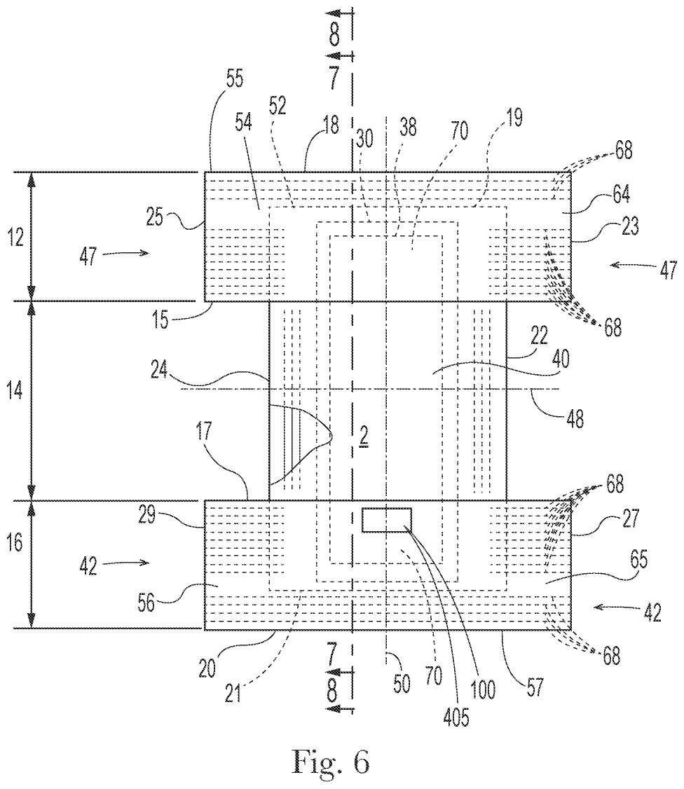

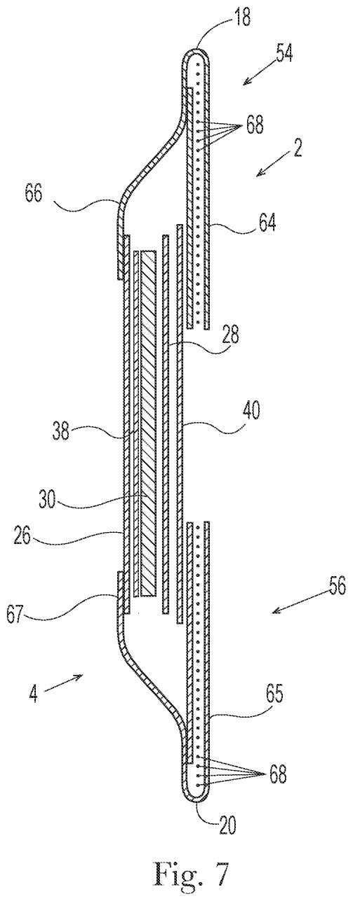

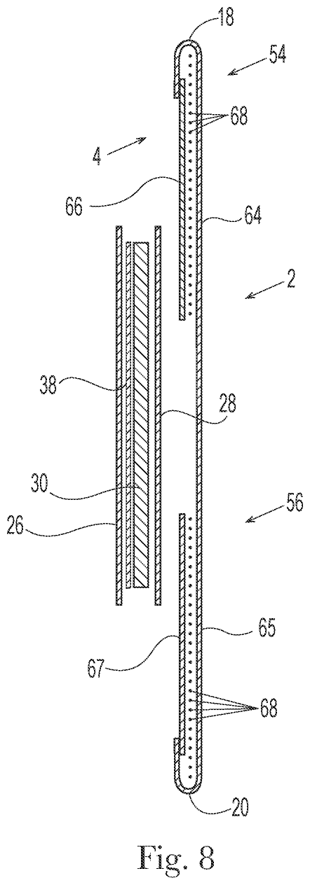

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52. In some embodiments, the back belt may comprise a nonwoven material and an elastic material.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Top Sheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material.

### Absorbent Core

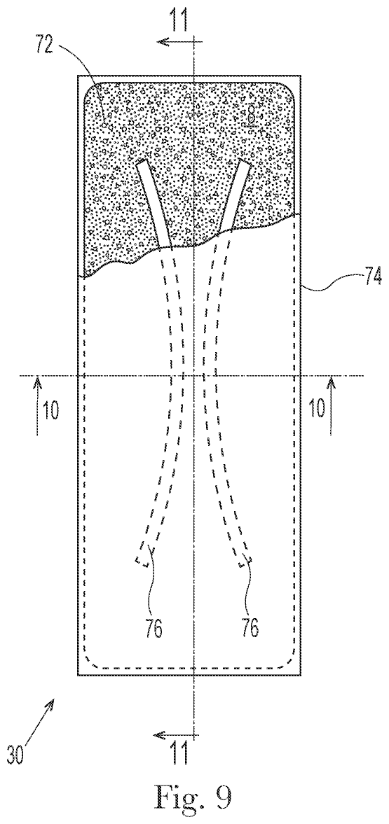

As used herein, the term “absorbent core” 30 refers to a component of the absorbent article 10 disposed in the article for absorbing and containing liquid such as urine received by the absorbent article. The absorbent core thus typically has a high absorbent capacity. An example absorbent core is schematically shown in FIGS. 9-11. The absorbent core comprises an absorbent material 72, that is typically enclosed within or sandwiched between a core bag 74.

The core wrap may be a single material that is folded and attached to itself, or it may comprise a separate top layer and bottom layer that may be bonded or otherwise joined together. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from g/g to 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. Appl. Pub. No. 2006/024433 (Blessing), U.S. Pat. Appl. Pub. No. 2008/0312617 and U.S. Pat. Appl. Pub. No. 2010/0051166A1 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate such as a carded nonwoven layer, having a porous structure into which SAP particles have been deposited, may also be used in present disclosure.

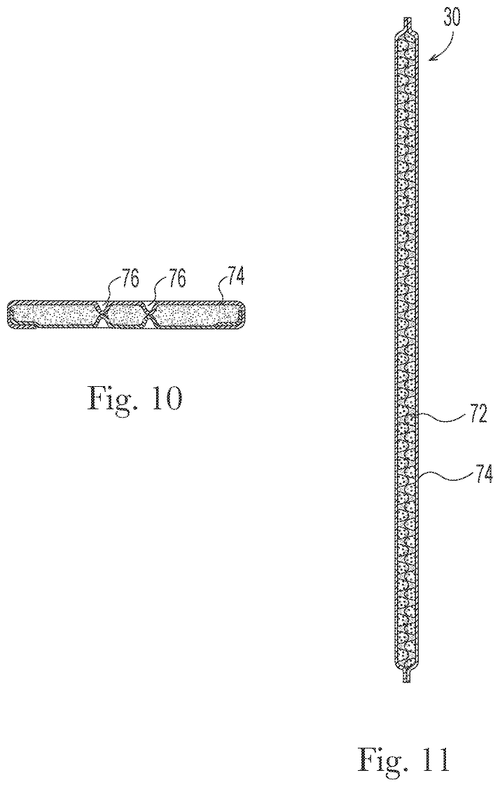

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/312,622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in WO2012/170778 (Rosati et al.) and US2012/0312491 (Jackels). Channels may of course also be formed in absorbent cores comprising a mix of cellulose fibers and SAP particles. These channels may embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper or in a refastenable pant context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g. color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Sanitary Napkin

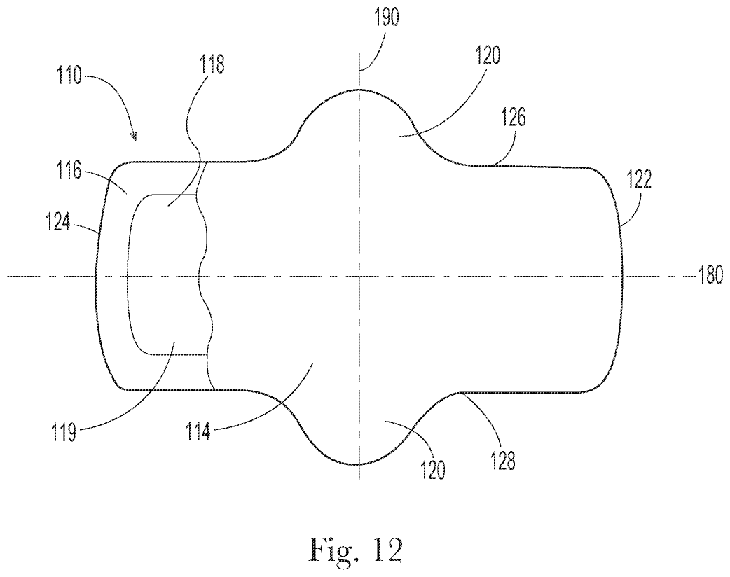

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Examples Cross-Sections of Absorbent Articles

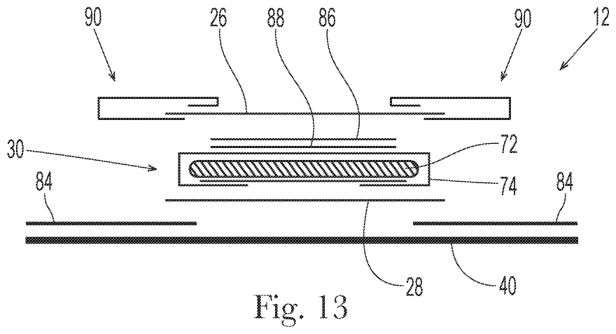

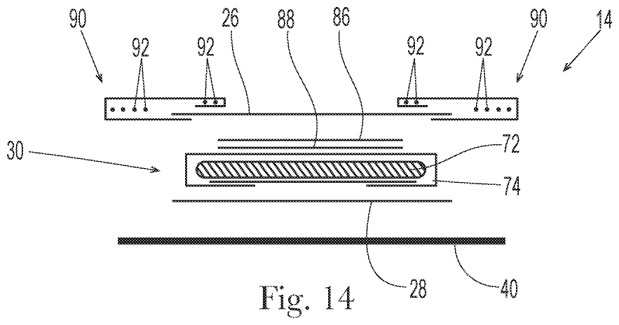

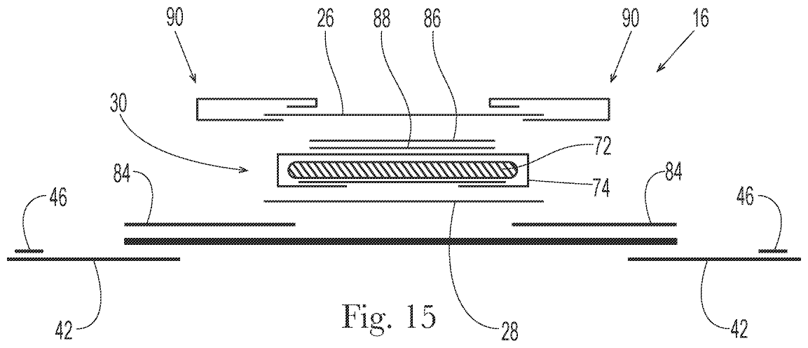

FIGS. 13-15 illustrate example cross-sectional views of absorbent articles within the scope of the present disclosure. FIG. 13 is an example cross-sectional view taken within a front waist region 12 of an absorbent article. FIG. 14 is an example cross-sectional view taken within a crotch region 14 of an absorbent article. FIG. 15 is an example cross-sectional view taken within a back waist region 16 of an absorbent article. In FIGS. 13-15, an outer cover material is element 40, a liquid permeable topsheet is element 26, opacity patches are elements 84, a liquid impermeable backsheet is element 28, an absorbent core is element 30, with the core bag being element 74, an absorbent material is element 72, and a distribution material is element 86. The distribution material 86 may comprise cross-linked cellulosic material and may be optional. An acquisition material is element 88. A liquid permeable topsheet is element 26. Barrier leg cuffs are elements 90. Elastics in the barrier leg cuffs are elements 92. Back ears are elements 42. Fasteners on the back ears 42 are elements 46. Construction glues and/or bonds between the various layers and/or components have been removed for clarity. Other cross-sectional configurations known to those of skill in the art are also within the scope of the present disclosure.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

In some aspects, the disposal fastener described herein may at least partially be comprised of bio-based content.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

### Disposal Fastener

Referring again to FIGS. 5-6, the absorbent articles of the present disclosure may comprise one or more disposal fasteners 100 for allowing absorbent article 10, or other absorbent articles, to be secured in a folded or rolled up configuration to contain the contents within the soiled article and provide for convenient disposal.

The absorbent article 10 may comprise a single disposal fastener or more than one disposal fastener, such as, a pair of disposal fasteners, three disposal fasteners, or four or more disposal fasteners. The disposal fastener may be disposed on the outer cover or may be attached to another absorbent article component such as a waistband, a belt, or a topsheet, for example. The disposal fastener may be joined to the outer cover or other absorbent article component mechanically and/or chemically. The disposal fastener may be mechanically joined by bonding, such as by heat, pressure or ultrasonics, to the outer cover or other absorbent article component. Further, the disposal fastener may be joined to the outer cover or other absorbent article component mechanically by hook and loop fasteners, such as Velcro®, or integrally formed fasteners. The disposal fastener may be chemically joined by, for example, an adhesive. It should be appreciated that the disposal fastener may be any suitable shape and size so long as it can engage at least a portion of the outer cover material or other absorbent article component and can hold the absorbent article in a compact configuration for disposal. For example, the fastener may be square, rectangular, circular, oval, heart shaped, semi-circular, triangular, or any other suitable shape.

The disposal fastener 100 may be disposed on the wearer-facing surface and/or the garment-facing surface. In some aspects, the disposal fastener is disposed on the garment-facing surface. The disposal fastener may be positioned in any suitable location on the absorbent article, including the front waist region 12, the back waist region 16, the crotch region 14, the back ears 42, the front ears 47, the front belt 54, and/or the back belt 56, so long as it is configured to secure the absorbent article in a compact configuration for disposal.

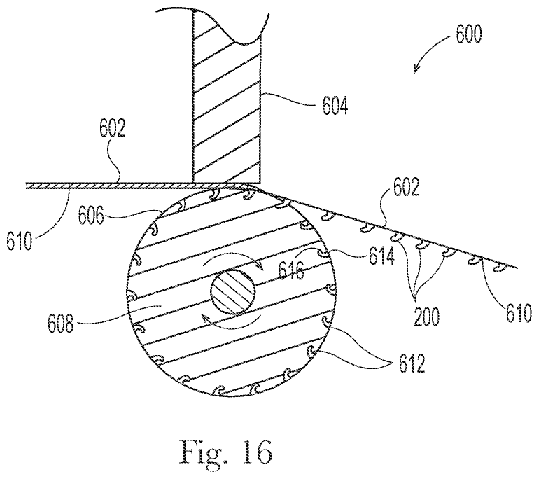

The disposal fastener 100 may comprise a plurality of integrally formed hook fasteners. Hook fasteners may be formed through any methods known in the art. FIG. 16 is a cross sectional view of a system 600 for forming hook fasteners 200 that are integral with a substrate 602, such as a nonwoven or a film, for example. As shown, the substrate 602 is disposed between a sonotrode 604 and a proximal surface 606 of a tool 608 such that a first side 610 of the substrate contacts the proximal surface of the tool 608. The tool 608 defines a plurality of cavities 612, each of the cavities 612 extending from a base 614 at the proximal surface to a distal end 616 within the tool to define a negative mold of a hook fastener. By way of example, FIG. 16 depicts the tool 608 as a rotating anvil, whereas other embodiments can include a tool with any of various shapes or configurations. The sonotrode 604 is configured to vibrate, for example at one or more ultrasonic frequencies, to locally heat the substrate 602 to cause the substrate to flow into the cavities. Thus, once the substrate is disposed between the sonotrode and the tool, ultrasonic energy is delivered from the sonotrode to the substrate such that a temperature of the first side of the substrate increases above the substrate's glass transition temperature. Thereafter or simultaneously, the substrate may be compressed between the sonotrode and the tool such that the substrate flows into the cavities until the substrate substantially fills the cavities to form a plurality of hook fasteners 200 on first side 610 of the substrate that are integral with the substrate. These hook fasteners may take on a number of geometries based on the shape of the cavities.

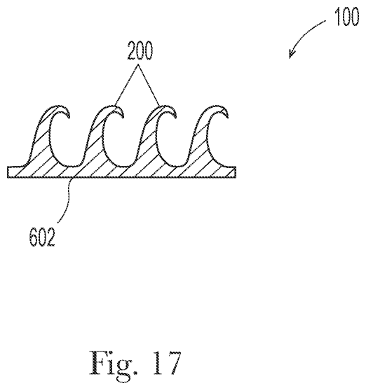

FIG. 17 shows a cross sectional view of a plurality of hook fasteners 200 that are integral with the substrate 602. The hook fasteners 200 may be any shape, such as nub, mushroom, or hook shaped, so long as they can engage with at least a portion of the outer cover material or other components of the absorbent article. Additional details of hook fastener shapes, and tools and methods for forming such hooks, are disclosed in U.S. Pat. No. 8,784,722. For example, FIGS. 2-2N of U.S. Pat. No. 8,784,722 depicts a plurality of hook fastener shapes.

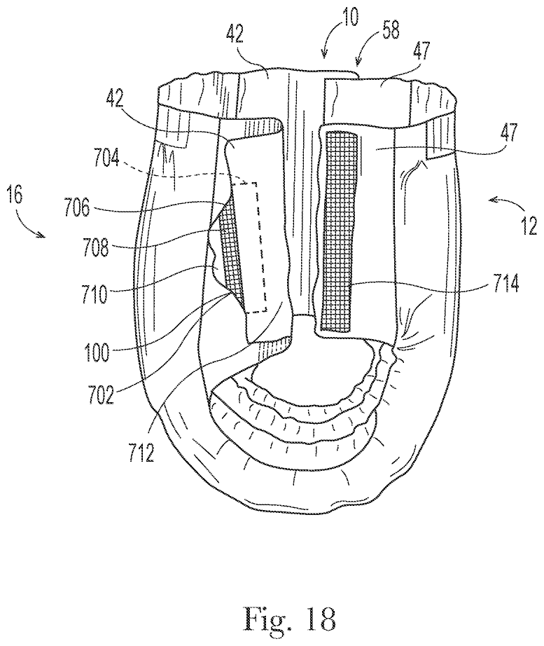

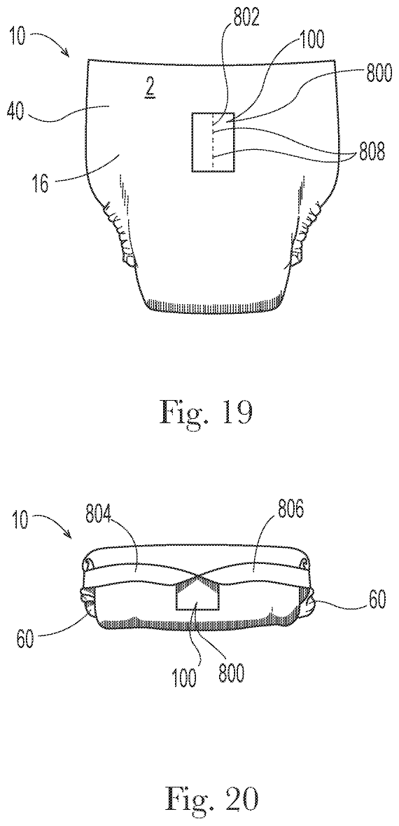

FIGS. 18-20 illustrate additional example absorbent articles 10 comprising one or more disposal fasteners 100. FIG. 18 is a side perspective of the absorbent article 10 wherein the disposal fastener 100 is a disposal tab 702 in an unfastened condition. FIGS. 19-20 are plan views of the absorbent article 10 wherein the disposal fastener 100 is a disposal tape 800.

Referring to FIG. 18, the absorbent article 10 may comprise back ears 42 extending from the back waist region 16 and comprise laterally opposite back joining portions 712 (also referred to herein as a first joining portion or a third joining portion). Absorbent article 10 may also comprise front ears 47 extending from front waist region and comprise laterally opposite front joining portions 714 (sometimes referred to as a second joining portion or a fourth joining portion). The back joining portions 712 may be configured for refastenable engagement to corresponding front joining portions 714. Back joining portion 712 of back ear 42 may be joined to front joining portion 714 of front ear 47 to form a refastenable side seam 58. A surface of each of the back and front joining portions 712, 714 may comprise a plurality of engaging elements. The engaging elements of the back joining portions 712 are adapted to repeatedly engage and disengage corresponding engaging elements of the front joining portions 714 to releasably secure absorbent article 10 in its three-dimensional wear configuration. The back and front joining portions 712, 714 may comprise any refastenable fasteners suitable for absorbent articles, such as adhesive fasteners, cohesive fasteners, mechanical fasteners, or the like. The back and front joining portions 712, 714 may comprise mechanical fastening components. Suitable mechanical fastening components may be provided by interlocking geometric shaped materials, such as hooks, loops, bulbs, mushrooms, arrowheads, balls on stems, male and female mating components, buckles, snaps, or the like. The back joining portions 712 (one on each side of the absorbent article 10) may comprise loop fasteners and the front joining portions 714 may comprise complementary hook fasteners. Alternatively, the back joining portions 712 may comprise hook fasteners and the front joining portions 714 may comprise complementary loop fasteners. The hooks fasteners may be integral with the back or front joining portions or may be manufactured from a portion of the back or front joining portions. Alternatively, the back and front joining portions 712, 714 may comprise interlocking similar surface fasteners, or adhesive and an adhesive-receptive landing zone or material, or hook fasteners and a nonwoven landing zone. When engaged, the back and front joining portions 712, 714 can define refastenable side seams 58.

The absorbent article 10 may further comprise one or more disposal fasteners 100, wherein the disposal fastener 100 is a disposal tab 702. In some aspects, the disposal tab 702 may be attached to each of the back ears 42 and extend in part transversely outward of the respective back ears for opposed relationship with the corresponding front ears in the wear configuration. In some aspects, the back ears 42 or front ears 47 may comprise a disposal tab extending beyond the back or front joining portions 712, 714. Each disposal tab 702 may comprise an attachment region 704 at which the disposal tab 702 is attached to the respective outer surface of the back or front ears 42, 47 of the absorbent article 10, and a fastener region 706 extending transversely outward from the attachment region 704. Alternatively, the disposal tab 702 may be integral with and form part of back ears 42 or front ears 47. The fastener region 706 of the disposal tab 702 may comprise a plurality of hook fasteners 708 that are integrally formed with a material of the disposal tab 702 for use in securing the absorbent article 10 in a compact disposal configuration. The disposal tab 702 may further comprise a grip region 710 transversely outward of the fastener region 706 for use in manually gripping and manipulating the disposal tab 702 relative to the absorbent article 10.

To dispose of the used absorbent article 10 after removal, the crotch region 14 and a portion of the back waist region 16 may be folded or rolled up over the front waist region 12. The disposal tabs 702 may then be gripped at grip regions 710 and pulled around the folded or rolled portion of the absorbent article 10 towards each other. The disposal tabs 702 may then engage a portion of the outer cover material, the back or front joining portions, or any other absorbent article component to secure the absorbent article in a compact configuration for disposal. This allows the absorbent article 10 to be relatively compact in the disposal configuration and to be held generally tightly in this configuration to reduce the risk of leakage of bodily exudates from the absorbent article.

Referring to FIG. 19, the absorbent article 10 may comprise a disposal fastener 100, wherein the disposal fastener 100 is a disposal tape 800. FIG. 19 illustrates the disposal tape 800 positioned on outer cover material 40 of absorbent article 10 in a folded pre-disposal configuration. The disposal tape 800 may be positioned anywhere on the absorbent article. In some aspects, the disposal tape 800 may be joined to the garment-facing surface 2 of the absorbent article 10. In some aspects, the disposal tape 800 may be joined to the garment-facing surface 2 of the absorbent article 10 in the back waist region 16.

The disposal tape 800 may comprise a frangible section 802. Alternatively, disposal tape 800 may be a unitary piece of material and does not comprise a frangible section. The frangible section 802 may provide for a separation of disposal tape 800 into generally independently movable strip portions 804, 806, as shown in FIG. 20. Frangible section 802 may be provided by selectively reducing the thickness of disposal tape 800 in a defined area to provide a line of weakness or puncturing the disposal tape 800 in a defined area to provide a line of perforations 808.

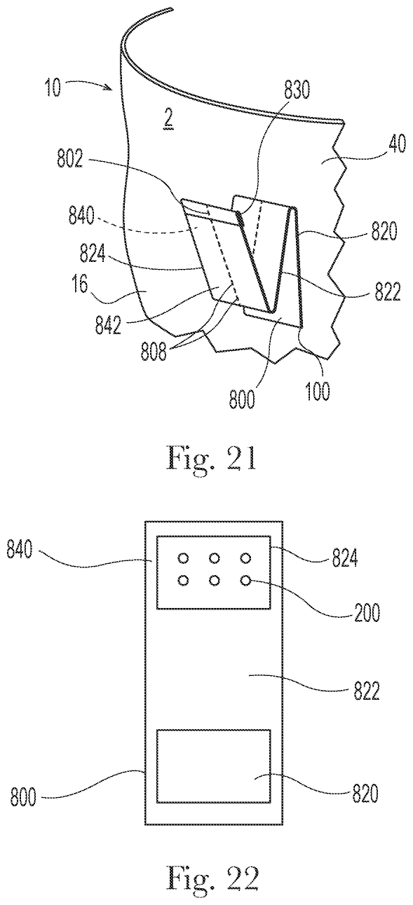

An enlarged illustration of the disposal tape 800 is shown in FIG. 21 in a partially extended configuration prior to separation along frangible section 802. Disposal tape 800 may comprise a first end region 820, a second end region 824, and a central region 822 disposed therebetween. The disposal tape 800 may also comprise grip tab 830 at the distal edge of second end region 824. In some aspects, the first end region 820 of disposal tape 800 may be joined to a portion of garment-facing surface 2. In some aspects, the central region 822 of the disposal tape 800 may be joined to a portion of the garment-facing surface 2. The disposal tape 800 may be bonded to the garment-facing surface by hook and loop fasteners, such as Velcro®, adhesive, or a patch of hooks integral with the garment-facing surface.

As shown in FIGS. 21 and 22, the disposal tape 800 may comprise a fastening surface 840 comprising a plurality of hook fasteners 200 and a backing surface 842 opposite the fastening surface 840. In some aspects, the fastening surface 840 of the second end region 824 may comprise a plurality of the hook fasteners 200 that are integral with a material of the second end region 824 of disposal tape 800. In some aspects, second end region 824 may comprise a second plurality of hook fasteners 200 that are integral with a material of the second end region 824 of disposal tape 800. The hook fasteners are configured to engage other portions of the absorbent article, such as the outer cover material, to secure the absorbent article in a compact configuration for disposal.

To use the disposal tape, the caregiver can unfold disposal tape 800 from its folded pre-disposal configuration by pulling on grip tab 830 and wrapping disposal tape 800 in a transverse or longitudinal direction around the absorbent article and engaging the plurality of hook fasteners with a portion of the outer cover material or other component of the absorbent article. FIG. 20 is an example illustration of absorbent article 10 in a compact disposal configuration with disposal tape 800 wrapped in a transverse direction around the absorbent article.

Disposal tape 800 may comprise one or more nonwoven materials and an elastic material. In some aspects, the elastic material may comprise an elastic film. In some aspects, the elastic material may comprise a plurality of elastic strands. The disposal tape 800 may comprise one or more layers of polyolefins. For example, that disposal tape may include one or more layers of polypropylene and/or one or more layers of polyester. The disposal tape may include one or more portions, such as previously discussed, and these one or more portions may be continuous or joined by an additional material, such as an adhesive, or joined by mechanical bonding.

Referring again to FIG. 5, in some embodiments, the disposal fastener 100 may be a disposal patch 405. The disposal patch 405 may be configured to engage and hold a portion of the garment-facing surface or other component of the absorbent article. The disposal patch 405 may be configured to engage and hold a portion of at least one of the back ear or the front ear as the caregiver rolls the absorbent article for disposal. The disposal patch 405 may be configured to engage and hold a portion of the central portion of the chassis as the caregiver rolls the absorbent article for disposal. The disposal patch 405 may be disposed in any suitable location on the article, including the garment-facing surface of the front waist region 12, back waist region 16, back ears 42, front ears 47, front belt 54, back belt 56 and/or crotch region 16, so long as it secures the absorbent article in a compact configuration for disposal. The disposal patch 405 may be covered, such as by a removable film, release paper, nonwoven, or as disclosed for example, in U.S. Patent Application No. 63/159,490, in order to prevent the hooks from catching on clothes.

The disposal patch 405 may comprise a plurality of integral hook fasteners 200 formed from a portion of outer cover material 40 or other components. The absorbent article may comprise one or more disposal patches 405 on a portion of outer cover material 40 or other components of absorbent article 10. Outer cover material 40 may be an outer cover nonwoven material. The absorbent article may comprise a second disposal patch comprising a second plurality of integral hook fasteners formed from a portion of the outer cover material or other components of absorbent article 10. The second disposal patch may be positioned on the front waist region or the back waist region and may be configured to engage and hold a portion of the garment-facing surface of the absorbent article. Each of the disposal patch and the second disposal patch may be positioned in the back waist region. Alternatively, the disposal patch may be positioned in the front waist region and the second disposal patch may be positioned in the back waist region. Alternatively, each of the disposal patch and the second disposal patch may be positioned in the front waist region. Disposal patches may also be positioned in other locations, such as the crotch region, for example.

While disposal fastener 100 is illustrated on a pant, it should be appreciated that the disposal fasteners described herein can be adapted to be used on a tape diaper as shown in FIGS. 1-3 or a sanitary napkin as shown in FIG. 12.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

### Arrays

“Array” means a display of packages comprising disposable absorbent articles of different article constructions (e.g., different elastomeric materials [compositionally and/or structurally] in the side panels, side flaps and/or belts flaps, different graphic elements, different product structures, fasteners or lack thereof). The packages may have the same brand and/or sub-brand and/or the same trademark registration and/or having been manufactured by or for a common manufacturer and the packages may be available at a common point of sale (e.g., oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Combinations

A. An absorbent article comprising a garment-facing surface; a wearer-facing surface; a front waist region; a back waist region; a crotch region positioned intermediate the front waist region and the back waist region; a first side edge; a second side edge opposite the first side edge; a first end edge; a second end edge opposite the first end edge; a liquid permeable topsheet forming a portion of the wearer-facing surface; a liquid impermeable backsheet; an absorbent core positioned at least partially intermediate the topsheet and the backsheet, wherein the absorbent core comprises an absorbent material; an acquisition material positioned intermediate the absorbent core and the topsheet; an outer cover nonwoven material in a face to face relationship with the backsheet and forming a portion of the garment-facing surface; and a disposal fastener comprising a plurality of integral hook fasteners.
B. The absorbent article according to paragraph A, comprising a first ear extending from the back waist region and comprising a first joining portion; a second ear extending from the front waist region and comprising a second joining portion; wherein the first joining portion of the first ear is configured to be joined to the second joining portion of the second ear to form a refastenable side seam.
C. The absorbent article according to paragraph B, wherein the disposal fastener is a disposal tab; wherein the first ear or the second ear comprises the disposal tab extending beyond the first or second joining portion; the disposal tab comprising a fastener region, the fastener region comprising a plurality of hook fasteners that are integrally formed with a material of the disposal tab.
D. The absorbent article according to paragraph C, further comprising a third ear extending from the back waist region and comprising a third joining portion; and a fourth ear extending from the front waist region and comprising a fourth joining portion; wherein the third joining portion of the third ear is configured to be joined to the fourth joining portion of the fourth ear to form a second refastenable side seam opposite the first refastenable side seam; the third ear or the fourth ear comprising a second disposal tab extending beyond the third or fourth joining portion; the second disposal tab comprising a second fastener region, the second fastener region comprising a plurality of hook fasteners that are integrally formed with a material of the second disposal tab.
E. The absorbent article according to paragraph A or B, wherein the disposal fastener is a disposal tape comprising a first end region, a second end region, and a central region, wherein the disposal tape comprises a plurality of hook fasteners in the second end region, and wherein the plurality of hook fasteners are integral with a material of the second end region of the disposal tape.
F. The absorbent article according to paragraph E, wherein the disposal tape is joined to the garment-facing surface in the back waist region.
G. The absorbent article according to paragraph E, wherein the first end region of the disposal tape is joined to a portion of the garment-facing surface.
H. The absorbent article according to paragraphs E-G, wherein the disposal tape comprises a nonwoven material and an elastic material.
I. The absorbent article according to paragraphs E-H, wherein the second end region of the disposal tape comprises a second plurality of hook fasteners, and wherein the second plurality of hook fasteners are integral with a material of the second end region of the disposal tape.
J. The absorbent article according to paragraph A or B, wherein the disposal fastener is a disposal patch comprising a plurality of hook fasteners formed from a portion of the outer cover nonwoven material, wherein the disposal patch is positioned on the front waist region or the back waist region, and wherein the disposal patch is configured to engage and hold a portion of the garment-facing surface of the absorbent article.
K. The absorbent article according to paragraph J, further comprising a second disposal patch comprising a second plurality of hook fasteners formed from a portion of the outer cover nonwoven material, wherein the second disposal patch is positioned in the front waist region or the back waist region, and wherein the disposal patch is configured to engage and hold a portion of the garment-face surface of the absorbent article.
L. The absorbent article according to paragraph K, wherein each of the disposal patch and the second disposal patch are positioned in the back waist region.
M. The absorbent article according to paragraph K, wherein the disposal patch is positioned in the front waist region and the second disposal patch is positioned in the back waist region.
N. The absorbent article according to paragraph K, wherein each of the disposal patch and the second disposal patch are positioned in the front waist region.
O. The absorbent article according to any of the preceding paragraphs, wherein the absorbent article is a pant.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a garment-facing surface;
a wearer-facing surface;
a front waist region;
a back waist region;
a crotch region positioned intermediate the front waist region and the back waist region;
a first side edge;
a second side edge opposite the first side edge;
a first end edge;
a second end edge opposite the first end edge;
a liquid permeable topsheet forming a portion of the wearer-facing surface;
a liquid impermeable backsheet;
an absorbent core positioned at least partially intermediate the topsheet and the backsheet, wherein the absorbent core comprises an absorbent material;
an acquisition material positioned intermediate the absorbent core and the topsheet;
an outer cover nonwoven material in a face to face relationship with the backsheet and forming a portion of the garment-facing surface; and
a disposal tape comprising a first end region, a second end region, and a central region, wherein the disposal tape comprises a plurality of hook fasteners in the second end region, and wherein the plurality of hook fasteners are integral with a material of the second end region of the disposal tape.

2. The absorbent article of claim 1, wherein the disposal tape is joined to the garment-facing surface in the back waist region.

3. The absorbent article of claim 1, wherein the absorbent article is a pant.

4. The absorbent article of claim 1, wherein the disposal tape comprises a nonwoven material and an elastic material.

5. The absorbent article of claim 4, wherein the elastic material comprises an elastic film.

6. The absorbent article of claim 4, wherein the elastic material comprises a plurality of elastic strands.

7. The absorbent article of claim 1, comprising a back belt positioned on the wearer-facing surface in the back waist region, wherein the back belt comprises a nonwoven material and an elastic material.

8. The absorbent article of claim 1, wherein the first end region of the disposal tape is joined to a portion of the garment-facing surface.

9. The absorbent article of claim 8, wherein the second end region comprises a second plurality of hook fasteners, and wherein the second plurality of hook fasteners are integral with a material of the second end region of the disposal tape.

10. The absorbent article of claim 1, wherein the central region of the disposal tape is joined to a portion of the garment-facing surface.

11. An absorbent article comprising:
a garment-facing surface;
a wearer-facing surface;
a front waist region;
a back waist region;
a crotch region positioned intermediate the front waist region and the back waist region;
a first side edge;
a second side edge opposite the first side edge;
a first end edge;
a second end edge opposite the first end edge;
a liquid permeable topsheet forming a portion of the wearer-facing surface;
a liquid impermeable backsheet;
an absorbent core positioned at least partially intermediate the topsheet and the backsheet, wherein the absorbent core comprises an absorbent material;
an acquisition material positioned intermediate the absorbent core and the topsheet;
an outer cover nonwoven material in a face to face relationship with the backsheet and forming a portion of the garment-facing surface;
a first ear extending from the back waist region and comprising a first joining portion; and
a second ear extending from the front waist region and comprising a second joining portion;
wherein the first joining portion of the first ear is configured to be joined to the second joining portion of the second ear to form a refastenable side seam;
the first ear or the second ear comprising a disposal tab extending beyond the first or second joining portion;
the disposal tab comprising a fastener region, the fastener region comprising a plurality of hook fasteners that are integrally formed with a material of the disposal tab.

12. The absorbent article of claim 11, comprising:
a third ear extending from the back waist region and comprising a third joining portion;
a fourth ear extending from the front waist region and comprising a fourth joining portion;
wherein the third joining portion of the third ear is configured to be joined to the fourth joining portion of the fourth ear to form a second refastenable side seam opposite the first refastenable side seam;
the third ear or the fourth ear comprising a second disposal tab extending beyond the third or fourth joining portion;
the second disposal tab comprising a second fastener region, the second fastener region comprising a plurality of hook fasteners that are integrally formed with a material of the second disposal tab.

13. The absorbent article of claim 11, wherein at least the first or second joining portion comprises a plurality of hook fasteners that are integrally formed with a material of the first ear or the second ear.

14. An absorbent article comprising:
a garment-facing surface;
a wearer-facing surface;
a front waist region;
a back waist region;
a crotch region positioned intermediate the front waist region and the back waist region;
a first side edge;
a second side edge opposite the first side edge;
a first end edge;
a second end edge opposite the first end edge;
a liquid permeable topsheet forming a portion of the wearer-facing surface;
a liquid impermeable backsheet;
an absorbent core positioned at least partially intermediate the topsheet and the backsheet, wherein the absorbent core comprises an absorbent material;
an acquisition material positioned intermediate the absorbent core and the topsheet;
an outer cover nonwoven material in a face to face relationship with the backsheet and forming a portion of the garment-facing surface; and
a disposal patch comprising a plurality of integral hook fasteners formed from a portion of the outer cover nonwoven material, wherein the disposal patch is positioned on the front waist region or the back waist region, and wherein the disposal patch is configured to engage and hold a portion of the absorbent article.

15. The absorbent article of claim 14, comprising a second disposal patch comprising a second plurality of integral hook fasteners formed from a portion of the outer cover nonwoven material, wherein the second disposal patch is positioned in the front waist region or the back waist region, and wherein the disposal patch is configured to engage and hold a portion of the absorbent article.

16. The absorbent article of claim 15, wherein each of the disposal patch and the second disposal patch are positioned in the back waist region.

17. The absorbent article claim 15, wherein the disposal patch is positioned in the front waist region and the second disposal patch is positioned in the back waist region.

18. The absorbent article of claim 15, wherein each of the disposal patch and the second disposal patch are positioned in the front waist region.

19. The absorbent article of claim 14, comprising:
a first ear extending from the back waist region and comprising a first joining portion;
a second ear extending from the front waist region and comprising a second joining portion;
wherein the first joining portion of the first ear is configured to be joined to the second joining portion of the second ear to form a refastenable side seam.

20. The absorbent article of claim 19, wherein the disposal patch is configured to engage and hold a portion of at least one of the first ear and the second ear.
